(19) United States
(12) Patent Application Publication
(10) Pub. No.: US 2012/0069960 A1
(43) Pub. Date: Mar. 22, 2012

(54) Portable radiation imaging system, portable radiation source holder used therein, and set of instruments for radiation imaging

(75) Inventors: Yusuke Kitagawa, Kanagawa (JP); Naoyuki Nishino, Kanagawa (JP)

(73) Assignee: FUJIFILM Corporation, Tokyo (JP)

(21) Appl. No.: 13/137,301

(22) Filed: Aug. 4, 2011

(30) Foreign Application Priority Data
Sep. 22, 2010 (JP) 2010-211862

(51) Int. Cl. A61B 6/02; G01N 23/04

(52) U.S. Cl. 378/41; 378/62; 378/194

(57) ABSTRACT

In an X-ray imaging system, an X-ray source has a lightweight and small-sized X-ray tube. The X-ray tube is a fixed anode X-ray tube without having a target rotating mechanism. The X-ray tube uses a cold cathode electron source, which does not need a filament and a heater. An image acquisition control device moves the X-ray source to each of plural positions predetermined on a cross bar of a holder by control of a drive source of a shift mechanism. Whenever the X-ray source reaches each position, the X-ray source emits X-rays to an object, and a cassette detects the X-rays transmitted through the object to take an image. Based on data of taken plural images, tomographic image, which puts emphasis on a region of interest inside the object, is produced.

### BACKGROUND OF THE INVENTION

1. Field of the Invention

The present invention relates to a portable radiation imaging system, a portable radiation source holder used therein, and a set of instruments for radiation imaging.

2. Description Related to the Prior Art

Tomosynthesis imaging is known as a technique for obtaining tomographic images to precisely inspect a region of interest (ROI) inside a human body. In this technique, while a radiation source e.g. an X-ray source (X-ray tube) is moved, X-rays are applied from the X-ray source to the human body at different angles to capture plural images. The obtained images are processed to yield the tomographic images, which put emphasis on desired tomographic planes (refer to U.S. Pat. No. 7,664,222).

U.S. Pat. No. 7,664,222 discloses a portable tomosynthesis imaging system suitably used in an emergency site, such as at an accident scene or at a scene of natural or other disaster. The system includes an X-ray source, an X-ray source holder, an X-ray image detector, a power supply, a control device, and a transporter. In performing the tomosynthesis imaging, all the components are carried by the transporter to the emergency site, and are assembled at the site. The U.S. Pat. No. 7,664,222 describes an embodiment of moving the single X-ray source to apply the X-rays at different angles to the human body, and an embodiment of using an array with plural X-ray sources.

Portability is of primary importance to such a portable tomosynthesis imaging system, allowing for easy carriage and an easy setup of the system. The most critical factor for determining the portability is the weight of the components constituting the system, particularly the weight of the X-ray source.

In the case of performing the tomosynthesis imaging with movement of the X-ray source, as described in the U.S. Pat. No. 7,664,222, if the X-ray source is heavy, the holder needs sturdiness enough to support the heavy X-ray source. The heavy X-ray source tends to cause vibration due to an acceleration force and an inertial force with its movement. The vibration causes a blur of the images, and impairs image quality. To prevent occurrence of the vibration, the holder needs to be heavy and stable. For this reason, the heavy X-ray source causes increase in the weight of the entire system and problems in the carriage and setup, resulting in a deterioration of the portability.

In spite of this fact, the U.S. Pat. No. 7,664,222 discloses neither the type of the X-ray source, nor the weight of the X-ray source and system.

### SUMMARY OF THE INVENTION

An object of the present invention is to provide a portable radiation imaging system with improved portability.

To achieve the above and other objects, a portable radiation imaging system according to the present invention includes a lightweight and small-sized portable radiation source, a portable radiation image detector, a portable holder, and a portable image processing device. The portable radiation source emits radiation to an object. The portable radiation image detector detects an image upon receiving the radiation transmitted through the object. The holder supports the radiation source in a movable manner relative to the radiation image detector. The portable image processing device processes data of plural images outputted from the radiation image detector. The radiation image detector produces each of the plural images by detecting the radiation, whenever the radiation is emitted from the radiation source situated at each of plural positions predetermined on the holder at different angles to the radiation image detector.

The radiation source preferably has a fixed anode radiation tube. The fixed anode radiation tube preferably uses a cold cathode electron source.

The portable radiation imaging system may further include a cable anchor for fastening a cable connected to the radiation source at a middle of the cable. A loose and excess portion of the cable may be contained in a cable cover in a sagging state between the cable anchor and the radiation source, or may be wound up and contained in the cable anchor.

The portable radiation imaging system preferably contains a shift mechanism for shifting the radiation source to the plural positions along a rail provided in the holder.

The portable radiation imaging system preferably carries out tomosynthesis imaging or stereoscopic imaging. In the tomosynthesis imaging, the data of the plural images is added to obtain a tomographic image in which a region of interest inside the object is emphasized. In the stereoscopic imaging, a stereo image is produced from the data of the plural images to provide a three-dimensional view.

The portable radiation imaging system preferably further includes a radiation shielding sheet attached to the holder. The radiation shielding sheet covers the radiation source, the radiation image detector, the holder, and a region of interest of the object.

A set of instruments for radiation imaging includes the portable radiation image detector, the portable holder, and the portable image processing device all of which are described above.

The portable holder according to the present invention includes a support section and a shift mechanism. The support section supports the radiation source. The shift mechanism shifts the support section to a plurality of positions, such that the radiation source emits the radiation at different angles to the radiation image detector.

According to the present invention, the portability of the radiation imaging system is ensured owing to the use of the lightweight and small-sized radiation source.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a perspective view of an X-ray imaging system;

FIG. 2 is a schematic sectional view of an X-ray tube;

FIG. 3 is a block diagram of an image acquisition control device;

FIG. 4 is an explanatory view of a tomosynthesis imaging process;

FIG. 5A is a schematic view showing a state of an X-ray source holder covered with an X-ray shielding sheet viewed from the right of a patient;

FIG. 5B is a schematic view showing a state of the holder covered with the X-ray shielding sheet viewed from the overhead of the patient; and

FIG. 6 is a perspective view of an X-ray source holder provided with a cable winder.

### DESCRIPTION OF THE PREFERRED EMBODIMENTS

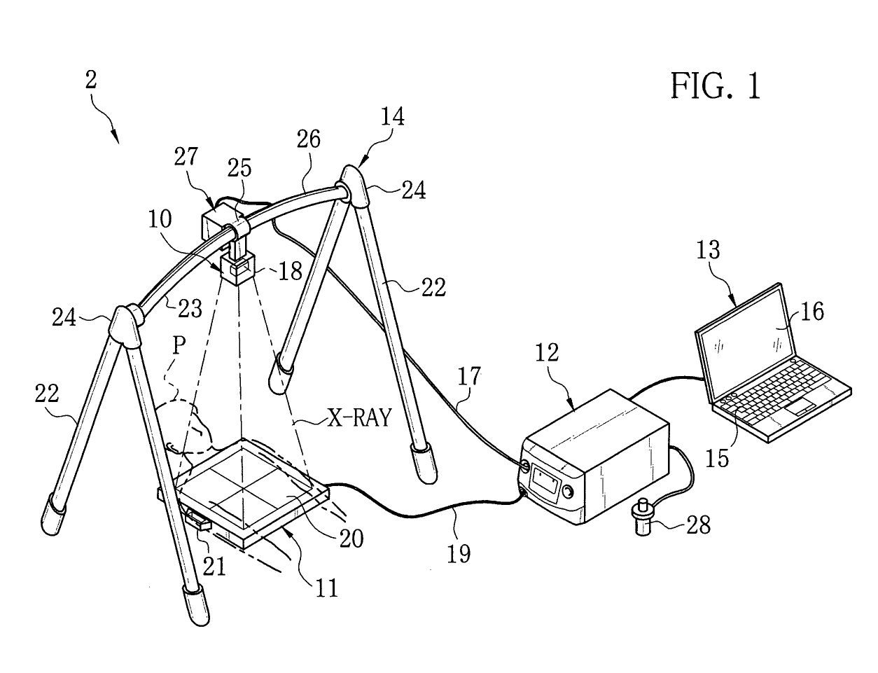

Referring to FIG. 1, an X-ray imaging system 2 is constituted of an X-ray source 10, a cassette 11, an image acquisition control device 12, a console 13, and an X-ray source holder (hereinafter simply called as holder) 14. The X-ray source 10 emits X-rays to a patient P. The cassette 11 detects the X-rays emitted from the X-ray source 10 and transmitted through the patient P, and outputs image data. The image acquisition control device 12 controls imaging operation of the X-ray source 10 and the cassette 11. The console 13 makes a setup of imaging conditions (tube voltage, tube current, exposure time, and the like of an X-ray tube 18 of the X-ray source 10) on the image acquisition control device 12. The holder 14 holds the X-ray source 10.

All of the X-ray source 10, the cassette 11, the image acquisition control device 12, the console 13, and the holder 14 are portable. To perform X-ray imaging, all these components are brought to a site requiring emergency medical treatment such as at an accident scene or at a scene of natural disaster, or to the bedside of a home-care patient.

The image acquisition control device 12 issues operation commands to the X-ray source 10 and the cassette 11 based on the imaging conditions inputted from an input device 15 of the console 13, so as to synchronize operation of the X-ray source 10 and the cassette 11. Upon receiving an exposure command signal from an exposure switch 28, the image acquisition control device 12 notifies the cassette 11 of the reception of the signal in order to synchronize the operation of the X-ray source 10 and the cassette 11.

The image data outputted from the cassette 11 is inputted to the console 13 through the image acquisition control device 12. The console 13 consists of a personal computer or a work station. The console 13 applies various types of image processing to the received image data, and displays a processed image on a monitor 16. The console 13 also outputs the image data to an external data storage device such as an image storage server.

The X-ray source 10 is connected to the image acquisition control device 12 with a cable 17, and receives electric power from the image acquisition control device 12. The X-ray source 10 has the X-ray tube 18 for emitting the X-rays by application of a high voltage from a driver 41 (high voltage generator, see FIG. 3), a collimator (irradiation field limiting unit, not shown) for limiting an irradiation field of the X-rays emitted from the X-ray tube 18, and the like.

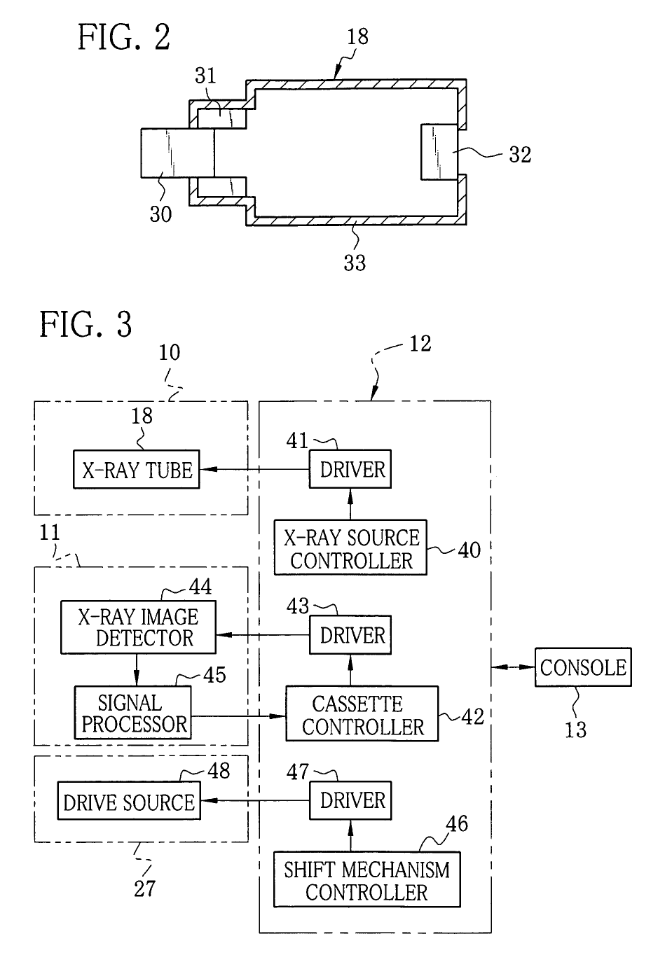

As shown in FIG. 2, the X-ray tube 18 is a fixed anode X-ray tube without having a target (anode) rotating mechanism. The X-ray tube 18 is constituted of a cold cathode electron source 30, an electron accelerator 31, a target 32, and a tube case 33. The cold cathode electron source 30 emits electrons. By collision of the electrons with the target 32, the X-rays are produced from the target 32. The tube case 33 contains the cold cathode electron source 30, the electron accelerator 31, and the target 32. Note that, the cold cathode electron source 30 needs neither a filament nor a heater for heating the filament, unlike a hot cathode.

Since the X-ray tube 18 has none of the target rotating mechanism, the filament, and the heater, the X-ray tube 18 is small in size and light in weight. Also, the X-ray tube 18 does not need time for preheating the filament, and allows quick application of the X-rays in response to the exposure command signal. Therefore, the X-ray tube 18 contributes to quickly starting the X-ray imaging even in case of emergency. As the X-ray tube 18, for example, there are available an ultraminiature X-ray generator contained in a coaxial cable having a diameter of 2 mm or less as described in Japanese Patent No. 3090910, or an X-ray tube using carbon nanostructures as described in “Development of Portable X-ray Sources Using Carbon Nanostructures” released from the National Institute of Advanced Industrial Science and Technology (AIST) on Mar. 19, 2009 (http://www.aist.go.jp/aist_e/latest_research/2009/20090424/2 0090424.html). The latter X-ray tube is driven by dry batteries. Thus, the X-ray source 10 may be disconnected from the image acquisition control device 12, and only the exposure command signal may be transmitted by radio.

As shown in FIG. 1, the cassette 11 is approximately in a rectangular shape. The cassette 11 is connected to the image acquisition control device 12 through a cable 19, and receives electric power from the image acquisition control device 12. The cassette 11 is appropriately placed under a patient's body in a position corresponding to a body part to be imaged, e.g. shoulder, knee, or the like with aiming an image receiving plane 20 at the X-ray source 10, as shown in FIG. 1. A grip 21 is provided on one side of the cassette 11 for ease of carriage. Note that, the X-ray source 10 and the image acquisition control device 12 have a carriage grip (not shown), just as with the cassette 11.

The cassette 11 contains an X-ray image detector 44 (see FIG. 3). The X-ray image detector 44 is a flat panel detector (FPD) having a matrix substrate composed of plural pixels arranged in two dimensions. Each of the pixels includes a thin film transistor (TFT) and an X-ray detecting element. Upon receiving the X-rays, each X-ray detecting element accumulates an electric charge, while the TFT is turned off. The amount of the accumulated electric charge depends on the intensity of the X-rays incident thereon. Thereafter, the TFT is turned on to read out the electric charge accumulated by the X-ray detecting element to outside. The readout electric charges are converted into a voltage signal by an integration amplifier of a signal processor 45. The converted voltage signal is subjected to an A/D conversion by an A/D converter of the signal processor 45 to produce digital image data.

The holder 14 has two pairs of support legs 22 erected on a floor or the ground with being open by a predetermined angle, and an arc-shaped cross bar 23. A triple joint 24 couples a top end of the single pair of support legs 22 to one end of the cross bar 23. Another triple joint 24 couples a top end of the other single pair of support legs 22 to the other end of the cross bar 23, so the holder 14 is assembled. To the cross bar 23, there is attached a connector 25, which establishes electrical and mechanical connection of the X-ray source 10. The connector 25 is connected to the cable 17. Thus, the X-ray source 10 attached to the connector 25 is electrically connected to the cable 17, and is hung from the cross bar 23. The X-ray source 10 emits the X-rays approximately in a vertical direction, while being hung from the cross bar 23.

The connector 25 is fitted onto a rail 26 formed into the shape of a groove in parallel with a longitudinal direction of the cross bar 23. The connector 25 is movable along the rail 26 by a shift mechanism 27. The shift mechanism 27 contains a drive source 48 (see FIG. 3) such as a stepping motor, which is driven at a command of the image acquisition control device 12. Upon actuating the drive source 48, the connector 25 and the X-ray source 10 connected to the connector 25 start moving along the rail 26. Upon stopping the drive source 48, the X-ray source 10 stops at a desired position on the cross bar 23. The image acquisition control device 12 counts the number of drive voltage pulses issued to the drive source 48 (the number of pulses applied to the stepping motor), and detects the position of the X-ray source 10 on the cross bar 23 based on the count number.

The image acquisition control device 12 controls operation of the drive source 48 to move the X-ray source 10 to plural positions (for example, forty to eighty positions) of the cross bar 23 predetermined in accordance with the imaging conditions. For example, the image acquisition control device 12 moves the X-ray source 10 stepwise from a left end to a right end of the cross bar 23. Whenever the X-ray source 10 reaches each predetermined position, the X-ray source 10 applies the X-rays to the body part of the patient P, and the cassette 11 detects the X-rays transmitted through the body part. Thus, the cassette 11 outputs image data of plural images detected while the X-ray source 10 changes its position a plural number of times and applies the X-rays from different angles. In this embodiment, the X-ray source 10 is moved along the rail 26 of the arc-shaped cross bar 23, but may be moved along a straight trajectory, instead of a curved trajectory, using a straight cross bar. In the case of the straight trajectory, the connector 25 may be provided with a swing mechanism to aim the X-ray source 10 at the cassette 11. The curved trajectory, as described in this embodiment, eliminates the need for providing the swing mechanism, because the X-ray source 10 faces toward the cassette 11 by itself.

In FIG. 3, an X-ray source controller 40 of the image acquisition control device 12 performs centralized control of the X-ray source 10. The X-ray source controller 40 controls operation of the X-ray tube 18 through a driver 41, so as to operate the X-ray tube 18 with established operation timing under the specified imaging conditions.

A cassette controller 42 performs centralized control of the cassette 11. The cassette controller 42 controls operation of the X-ray image detector 44 of the cassette 11 through a driver 43, so as to operate the X-ray image detector 44 with established operation timing. Also, the cassette controller 42 receives the image data from a signal processor 45 having the integration amplifier and the A/D converter, and transfers the image data to the console 13.

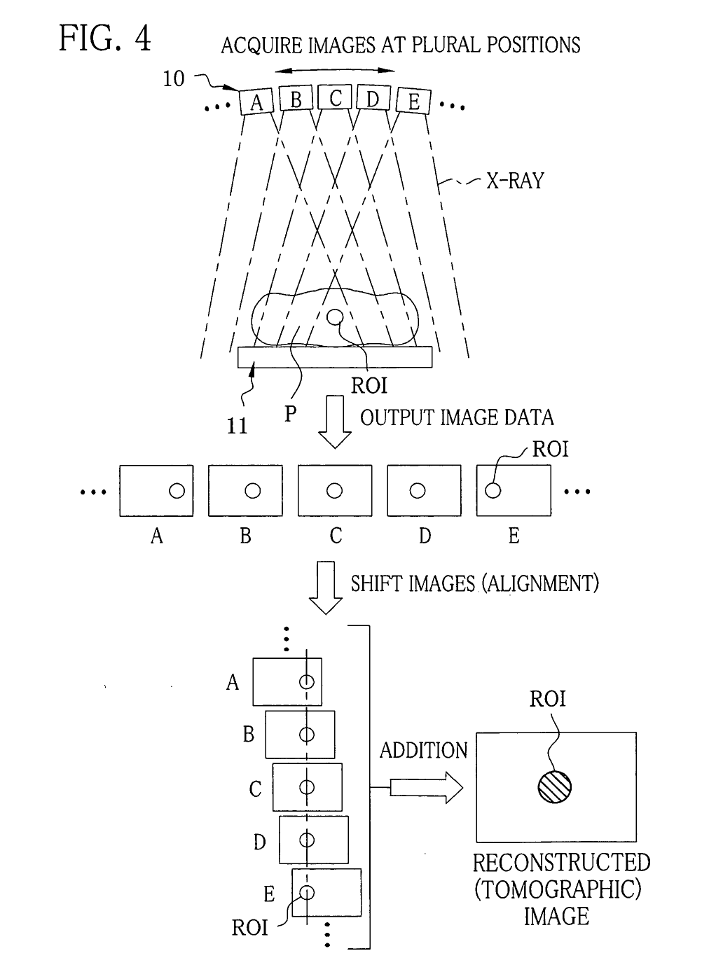

As schematically shown in FIG. 4, the console 13 produces tomographic images of the patient P, more specifically, the tomographic images (also referred to as reconstructed images) parallel to the image receiving plane 20 of the cassette 11 at a region of interest (ROI) inside the body part of the patient P, based on the image data of the plural images outputted from the cassette 11 while the X-ray source 10 changes its position a plural number of times and applies the X-rays from the different angles. In a method for producing the tomographic image, for example, images captured at different positions A to E are subjected to shift processing to align the ROI among the images. After the shift processing, the images are subjected to addition processing to obtain the reconstructed image, in which the ROI is emphasized.

Another method for producing the tomographic image, for example, a simple back projection method or a filtered back projection method may be adopted instead. In the simple back projection method, a plurality of images are back projected without being applied to a reconstruction filter, and then are subjected to addition processing to obtain a reconstructed image. On the other hand, the filtered back projection method includes two ways. In one way, a plurality of images are back projected after being applied to a reconstruction filter as a convolution filter. Then, the projected images are subjected to addition processing to obtain a reconstructed image. In the other way, a plurality of images are temporarily converted into frequency space data by Fourier transformation. The frequency space data is applied to a reconstruction filter, and is back projected, and thereafter is subjected to addition processing to obtain a reconstructed image.

Referring to FIG. 3, a shift mechanism controller 46 controls the operation of the drive source 48 of the shift mechanism 27 through a driver 47. The X-ray source controller 40, the cassette controller 42, and the shift mechanism controller 46 cooperate to perform tomosynthesis imaging, by which the image data of the plural images is obtained while the X-ray source 10 changes its position a plural number of times and applies the X-rays from the different angles, and the reconstructed images are produced from the image data.

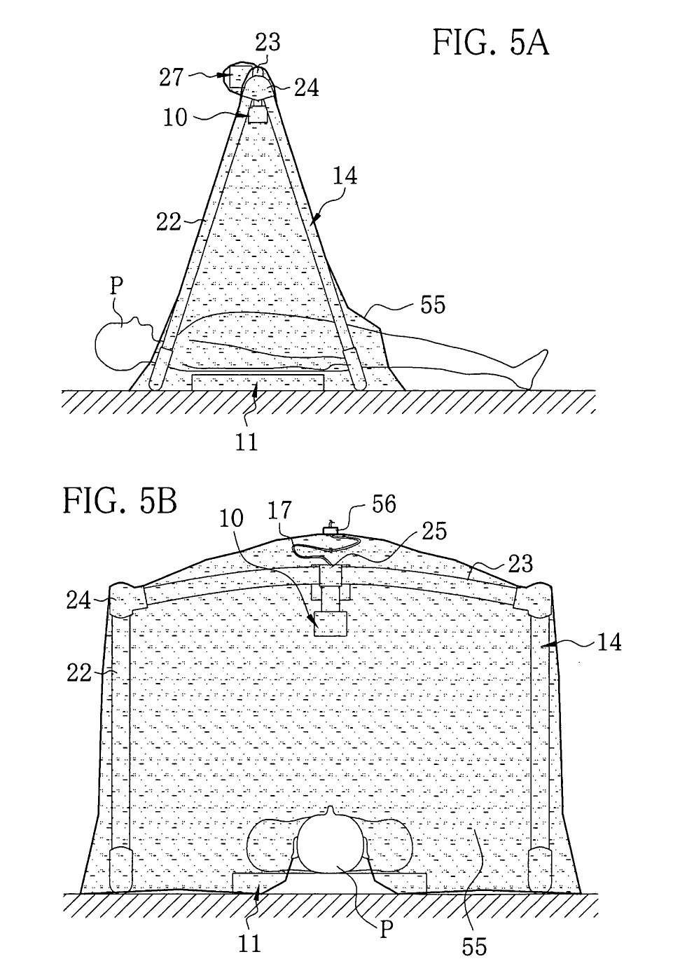

As shown in FIGS. 5A and 5B, when the X-ray imaging system 2 performs the X-ray imaging, the holder 14 is covered with an X-ray shielding sheet 55, for the purpose of preventing exposure of unnecessary body parts or persons to the X-rays. FIG. 5A shows a state viewed from the right of the patient P. FIG. 5B shows a state viewed from the overhead of the patient P.

The X-ray shielding sheet 55 is made of foldable cloth containing an X-ray shielding material such as lead. The X-ray shielding sheet 55 is the size of covering almost the entire X-ray imaging system 2. The X-ray shielding sheet 55 is attached to the joints 24 and the cross bar 23, and hangs down to the bottom of the support legs 22. Thus, the X-ray shielding sheet 55 covers the entire X-ray imaging system 2 together with the body part to be imaged of the patient P. For attachment of the X-ray shielding sheet 55, hooks, snaps, hook-and-loop fasteners, clamping screws, or the like are available.

A cable anchor 56 is provided on the middle of a top surface of the X-ray shielding sheet 55. The cable anchor 56 fastens the cable 17 at its middle between the image acquisition control device 12 and the connector 25. The cable 17 is placed under the X-ray shielding sheet 55 with sagging between a rear surface of the X-ray shielding sheet 55 and the cross bar 23 of the holder 14. The cable 17 sags in such a degree as not to be tautly stretched, even if the connector 25 moves from one end of the cross bar 23 to the other end thereof along the rail 26. The cable 17 is made straight and curved with a support of the cable anchor 56, while the connector 25 is moving.

Next, operation of the X-ray imaging system 2 will be described. A radiologist who uses the X-ray imaging system 2 carries a set of the X-ray imaging system 2 to a site requiring the X-ray imaging. The radiologist assembles the holder 14 as shown in FIG. 1, and couples the X-ray source 10 to the connector 25. The radiologist connects the cable 17 to the connector 25, and connects the cable 19 to the cassette 11. Thus, the X-ray source 10, the cassette 11, and the image acquisition control device 12 are connected to one another.

After completion of a setup of the X-ray imaging system 2, the patient P lies down in an appropriate position between the X-ray source 10 and the cassette 11. The X-ray shielding sheet 55 is attached to the holder 14 so as to cover the body part to be imaged of the patient P and the entire X-ray imaging system 2. The cable anchor 56 fastens the middle of the cable 17. The cable 17 is placed under the X-ray shielding sheet 55 with sagging at a portion led out of the cable anchor 56.

The radiologist inputs the imaging conditions and the like from the input device 15 of the console 13, and enters an exposure start command by a press of the exposure switch 28 connected to the image acquisition control-device 12. In response to the exposure start command, the image acquisition control device 12 actuates the drive source 48 of the shift mechanism 27, such that the X-ray source 10 is moved to the plurality of positions predetermined on the cross bar 23. Under control of the image acquisition control device 12, whenever the X-ray source 10 reaches each predetermined position, the X-rays are applied from the X-ray tube 18 of the X-ray source 10 to the body part of the patient P, and the X-ray image detector 44 of the cassette 11 detects the X-rays transmitted through the body part.

The console 13 produces the reconstructed images based on the plurality of images obtained as above. The reconstructed images are displayed on the monitor 16. The radiologist carries out a proper procedure, e.g. makes a diagnosis by observation of the reconstructed image displayed on the monitor 16, or sends image data of the reconstructed images through a network to a specialist in a remote medical hospital or center to seek advice from the specialist.

As described above, according to the present invention, the X-ray tube 18 is light in weight and small in size. Thus, the X-ray tube 18 is easy to carry and assemble, and relieves a burden of the radiologist. The lightweight X-ray tube 18 eliminates the need for imparting sturdiness to components of the holder 14, and is able to reduce the weight of the holder 14 too.

The lightweight and small-sized X-ray tube 18 prevents occurrence of vibration caused by movement of the X-ray source 10. This eliminates the need for instituting complicated anti-vibration measures using a vibration detector, such as interruption of imaging operation until the vibration subsides, or correction of the images based on a vibration detection result. Therefore, the imaging operation can be carried out smoothly and speedily. Also, it is possible to minimize a deterioration of image quality and a blur in the image due to the vibration, and obtain the sharp images.

An X-ray tube disclosed in the Japanese Patent No. 3090910 trades off a dose of the X-rays for small size and light weight. However, a small dose of the X-rays is contrarily suitable for the tomosynthesis imaging described in this embodiment, because the images are taken with the small dose of the X-rays while the X-ray source 10 is moving.

The present invention is applicable to stereoscopic imaging, in addition to the tomosynthesis imaging. In the stereoscopic imaging, the X-ray source 10 is moved to at least two positions of different viewpoints, and imaging operation is carried out at each position. A stereo image is obtained from images obtained each position, and forms a three-dimensional view.

The X-ray shielding sheet 55 covers the body part to be imaged of the patient P and the X-ray imaging system 2. This prevents a leak of the applied X-rays to outside, and reduces the risk of unnecessary X-ray exposure of a person around the X-ray imaging system 2. Since the patent P puts his/her body, including head and legs, outside space enclosed by the X-ray shielding sheet 55 other than the body part to be imaged, it is possible to prevent the risk of excessive X-ray exposure of the patient P other than the body part to be imaged. The X-ray shielding sheet 55 facilitates using the X-ray imaging system 2 without hesitation even in crowded space.

The X-ray shielding sheet 55 is folded or rolled when unused. For example, the cross bar 23 is made hollow, and the X-ray shielding sheet 55 may be contained in a hollow of the cross bar 23.

The cable 17 is fastened by the cable anchor 56 at its middle, and is placed under the X-ray shielding sheet 55 with sagging at the portion led out of the cable anchor 56. Therefore, the cable 17 is made compact. This prevents occurrence of an accident that a person trips over the cable 17 and falls, or an accident that the cable 17 is accidentally pulled out and the imaging operation is interrupted. Likewise, the cable 19 connecting the cassette 11 to the image acquisition control device 12 may be placed under the X-ray shielding sheet 55 with sagging.

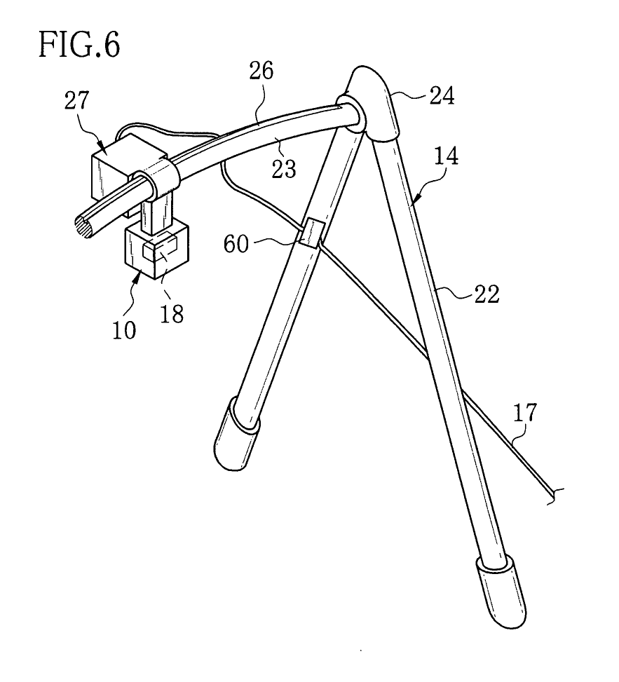

To prevent the accident due to the cable 17 and improve containment and portability properties of the cable 17, the cable 17 may be provided with a winder 60, as shown in FIG. 6. The cable 17 is wound up by the winder 60 when unused. The cable 17 is pulled out of the winder 60 as needed, when used. The winder 60 may be attached to the support leg 22, as shown in FIG. 6. In another case, a winder may be provided in the image acquisition control device 12 to wind up the cable 17 into the image acquisition control device 12. In further another case, the cable anchor 56 described in the above embodiment may have the function of winding up the cable 17 jutting out the X-ray shielding sheet 55.

Especially, in the case of using the X-ray imaging system 2 at the bedside of the home-care patient, the cable can be caught on an unforeseen object such as personal belongings, unlike in a hospital. In this case, the imaging operation may be carried out while the caught cable holds back the X-ray source 10 in a certain position, or the caught cable may impose an excessive burden on the drive source 48 and cause a breakdown of the X-ray imaging system 2. Thus, the catch of the cable may cause a serious problem. To solve this problem, the cable is fastened or wound up in the present invention. Limiting a movable range of the cable is very effective at ensuring security.

Note that, the X-ray imaging system according to the present invention is not limited to above embodiments, and is modified into various configurations within the scope of the present invention.

For example, in the above embodiment, the X-ray image detector 44 is actuated in response to the command from the image acquisition control device 12. However, the X-ray image detector 44 may detect application of the X-rays by itself. In this case, the X-ray image detector 44 may be actuated without the command from the image acquisition control device 12.

The X-ray image detector 44 is not limited to of a direct conversion type, as is described in the above embodiment, but may be of an indirect conversion type. In the indirect conversion type of X-ray image detector, the incident X-rays are temporarily converted into visible light by a scintillator, and the visible light is converted into an electric signal using a solid-state detecting element such as amorphous silicon (a-Si).

The cassette 11 is connected to the image acquisition control device 12 with the cable 19, but may be connected by radio. In the case of connection by radio, the cassette 11 is equipped with a battery for electric power supply.

The cassette 11 may have the functions of the cassette controller 42 and the driver 43, and the shift mechanism 27 may have the functions of the shift mechanism controller 46 and the driver 47, instead that the image acquisition control device 12 has the cassette controller 42, the shift mechanism controller 46, and the drivers 43 and 47. Additionally, the driver 41 being the high voltage generator may be separated from the image acquisition control device 12.

The image acquisition control device 12 may produce the reconstructed image, instead of the console 13. The X-ray source 10 may be moved manually without using a driving force of the drive source 48. The cassette 11 may be provided with a moving mechanism to synchronously move the cassette 11 in a direction opposite to a moving direction of the X-ray source 10.

The support legs 22 and the cross bar 23 may be stretchable and shrinkable, so that the width between the support legs 22 and the distance (SID: source image distance) between the X-ray source 10 and the image receiving plane 20 become adjustable. The support legs 22 and the cross bar 23 may be folded up into a single piece, using fold-up joints.

A maximum projection angle of the X-ray source 10 defined by an opening of the collimator is approximately of the order of 12°, in most cases. To increase or decrease the size of an irradiation field of the X-rays without changing the size of the opening of the collimator, the SID is adjusted by shrinking or stretching the support legs 22. Note that, the maximum projection angle refers to a vertex angle of an isosceles triangle, which is formed when a focus of the X-ray tube 18 is defined as a vertex and a straight line connecting both ends of the opening is defined as a base.

The X-ray shielding sheet 55 may not be a size of covering the entire system, as is described in the above embodiments, but may be a size of containing at least the sagging cable 17. Separately from the X-ray shielding sheet 55, a cover for containing the cable 17 may be provided.

The present invention is applicable to an imaging system using not only the X-rays but also other types of radiation such as γ-rays.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A portable radiation imaging system comprising:
a lightweight and small-sized portable radiation source for emitting radiation to an object;
a portable radiation image detector for detecting an image upon receiving said radiation transmitted through said object;
a portable holder for supporting said radiation source in a movable manner relative to said radiation image detector; and
a portable image processing device for processing data of plural images outputted from said radiation image detector; and
wherein said radiation image detector produces each of said plural images by detecting said radiation, whenever said radiation is emitted from said radiation source situated at each of plural positions predetermined on said holder at different angles to said radiation image detector.

2. The portable radiation imaging system according to claim 1, wherein said radiation source has a fixed anode radiation tube.

3. The portable radiation imaging system according to claim 2, wherein said fixed anode radiation tube uses a cold cathode electron source.

4. The portable radiation imaging system according to claim 1, further comprising:
a cable anchor for fastening a cable connected to said radiation source at a middle of said cable.

5. The portable radiation imaging system according to claim 4, further comprising:
a cable cover for containing a loose and excess portion of said cable in a sagging state between said cable anchor and said radiation source.

6. The portable radiation imaging system according to claim 4, wherein said cable anchor winds up and contains a loose and excess portion of said cable.

7. The portable radiation imaging system according to claim 1, further comprising:
a shift mechanism for shifting said radiation source to said plural positions along a rail provided in said holder.

8. The portable radiation imaging system according to claim 1, wherein tomosynthesis imaging is carried out by adding said data of said plural images, to obtain a tomographic image in which a region of interest inside said object is emphasized.

9. The portable radiation imaging system according to claim 1, wherein stereoscopic imaging for providing a three-dimensional view is carried out to produce a stereo image from said data of said plural images.

10. The portable radiation imaging system according to claim 1, further comprising:
a radiation shielding sheet attached to said holder.

11. The portable radiation imaging system according to claim 10, wherein said radiation shielding sheet covers said radiation source, said radiation image detector, said holder, and a region of interest of said object.

12. A set of instruments for radiation imaging comprising:
a portable radiation image detector for detecting an image upon receiving radiation having been transmitted through an object;
a portable holder for supporting a lightweight and small-sized radiation source in a movable manner relative to said radiation image detector, said radiation source emitting said radiation to said object; and
a portable image processing device for processing data of plural images outputted from said radiation image detector; and
wherein said radiation image detector produces each of said plural images by detecting said radiation, whenever said radiation is emitted from said radiation source situated at each of plural positions predetermined on said holder at different angles to said radiation image detector.

13. A portable radiation source holder used in a portable radiation imaging system, said portable radiation imaging system including a lightweight and small-sized portable radiation source for emitting radiation to an object and a portable radiation image detector for detecting an image upon receiving said radiation transmitted through said object, said portable radiation source holder comprising:
a support section for supporting said radiation source; and
a shift mechanism for shifting said support section to a plurality of positions such that said radiation source emits said radiation at different angles to said radiation image detector.
